(19) United States
(12) Patent Application Publication
(10) Pub. No.: US 2006/0160168 A1
(43) Pub. Date: Jul. 20, 2006

(54) Methods and products for identifying compounds that modulate cell plasma membrane repair

(75) Inventors: Robert H. Brown, Needham, MA (US); Niall J. Lennon, Tullow (IE)

Correspondence Address: WOLF GREENFIELD & SACKS, PC;NULL, FEDERAL RESERVE PLAZA, 600 ATLANTIC AVENUE, BOSTON, MA, 02210-2206, US

(73) Assignee: The General Hospital Corporation, Boston, MA (US)

(21) Appl. No.: 11/330,906

(22) Filed: Jan. 12, 2006

Related U.S. Application Data
(60) Provisional application No. 60/643,800, filed on Jan. 14, 2005.

(51) Int. Cl. C12Q 1/26

(52) U.S. Cl. 435/25

(57) ABSTRACT

The invention relates in some aspects to methods and kits for determining plasma membrane repair status in cells, tissues, and subjects. The methods include, in part, the use of high-throughput assays for assessing cell plasma membrane repair status. The methods and kits of the invention are useful to identify compounds that enhance or inhibit plasma membrane repair and to identify plasma membrane repair-associated disorders and diseases in cells, tissues, and subjects. The methods of the invention are also useful to select treatments, monitor treatment efficacy, and to assess the onset, progression, and/or regression of plasma membrane repair-associated disorders and diseases.

### RELATED APPLICATIONS

This application claims priority under 35 U.S.C. §119 from U.S. provisional application Ser. No. 60/643,800, filed Jan. 14, 2005, the entire content of which is incorporated herein in its entirety.

### GOVERNMENT SUPPORT

This invention was made in part with government support under grant number 5PO1NS40828-02 from the National Institute of Neurological Disorders and Stroke (NINDS). The government may have certain rights in this invention.

### FIELD OF THE INVENTION

The invention relates in some aspects to methods and kits for determining plasma membrane repair status in cells, tissues, and subjects and identifying compounds that enhance or inhibit plasma membrane repair.

### BACKGROUND OF THE INVENTION

Mechanisms of cell plasma membrane repair are important to allow cells to maintain their integrity. Defects or disruption in plasma membrane repair have been associated with a number of diseases and disorders such as muscular dystrophies, metabolic disorders, and cell and tissue injury.

The muscular dystrophies represent a large group of progressive hereditary diseases that have traditionally been classified using clinical criteria including pattern of muscle involvement, age of onset and mode of inheritance (Laval, S. H. et al., Neuropathol. Appl. Neurobiol. 30(2): 91-105, 2004). Most muscular dystrophies are characterized by common pathological features such as myofiber breakdown, inflammation and muscle regeneration. Recent work has elucidated a new and important pathway in skeletal muscle biology, the process of muscle membrane repair (Lennon, N. J. et al., Biol. Chem. 278(5): 50466-50473, 2003; Bansal, D. et al., Trends Cell Bio. 14(4): 206-213, 2004). Mutations in the muscular dystrophy protein, dysferlin, are the first to be identified as causing a defect in the repair process. Limb girdle muscular dystrophy type 2B (LGMD2B) and Miyoshi Myopathy (MM) result from mutations in the dysferlin gene (Bashir, R. et al., Nat Genet. 20(1): 37-42, 1998; Liu, J. et al., Nat Genet. 20(1): 31-36, 1998).

Currently no effective therapies exist for these conditions and researchers lack the tools to look for such therapies in an efficient and reproducible way, i.e., high-throughput drug screening assays in multi-well plates. Available methods to examine membrane repair include strategies to induce injury in cells followed by analysis of membrane repair. Chemical and mechanical methods are used to induce membrane injury. Mechanical strategies include stretching cells plated on flexible surfaces, laser irradiation, and cell membrane wounding with finely pulled capillary glass. Following membrane wounding, the rate of recovery of the cell membrane is determined using various dyes and toxins that distinguish between wounded and repaired cells. These currently available injury-inducing methods do not permit high-throughput testing of plasma membrane repair.

There is a lack of available tools that allow efficient, reproducible, and reliable monitoring of the repair of cell membranes in a high-throughput manner. Thus, there is a need for improved methods of monitoring cell plasma membrane repair and for high-throughput screening methods that can be used to identify compounds useful for treatment of neuromuscular diseases as well as other diseases and disorders.

### SUMMARY OF THE INVENTION

We have discovered methods of monitoring and assessing cell plasma membrane repair and methods of inducing plasma membrane repair. The methods allow simultaneous examination of plasma membrane repair in one or more cell samples thus permitting high-throughput evaluation of plasma membrane repair status. The invention, in part, includes methods of assessing candidate pharmaceutical agents for their ability to modulate plasma membrane repair in cells. In addition, the invention also includes, in part, methods to determine whether a cell has a plasma membrane repair-associated disease, disorder, or condition. Thus, the invention can be used to determine if a cell of interest has normal or abnormal plasma membrane repair. Cells of interest may be cells from a subject for whom a diagnosis is desirable, genetically modified cells, disease-model cells, etc. The methods of the invention also allow diagnostic assessment of cell samples that can be used for initial diagnosis and/or for monitoring treatment strategies in cells and subjects.

The invention includes, in part, screening methods for the identification of existing drug molecules and novel therapeutic agents with an effect on cell plasma membrane repair.

The screening methods of the invention include, but are not limited to, high-throughput screening methods. The invention, in part, relates to a novel membrane-injury paradigm coupled with various assay endpoint determinations to monitor plasma membrane repair status. The membrane injury aspects of the invention, in part, include the use of a device to injure a cell plasma membrane. The device (e.g. floating pin) can be contacted with a cell plasma membrane one or more times, which results in injury to the cell plasma membrane.

The device useful in the methods of the invention is contacted with a cell plasma membrane and the force the device exerts on the plasma membrane is about equal to the weight of the device under gravity. An example of a device useful in the methods of the invention is a floating pin.

A number of assay endpoints can be used to determine the repair of the plasma membrane of a cell injured with the methods of the invention. The assay endpoints of the invention are useful in rapid screening and high-throughput screening and in some embodiments, the assay endpoints are microplate reader-compatible endpoints. The invention is applicable in drug discovery for diseases in which altered membrane repair is implicated, such as limb girdle muscular dystrophy type 2B (LGMD2B) and Miyoshi myopathy (MM). The methods and kits of the invention are useful to monitor cell plasma membrane repair. Additionally, the methods and kits of the invention can be used to identify drug molecules or chemical entities that alter the level or rate of membrane repair. The methods and kits can also be used to identify cell membrane injury-associated disorders and/or diseases in cell, tissues, and/or subjects.

According to one aspect of the invention, methods for identifying compounds that modulate cell plasma membrane repair are provided. The methods include injuring a plasma membrane of a cell by contacting the cell with a device that exerts force on the cell equal to about the weight of the device under gravity, contacting the injured cell with a candidate compound, and measuring the repair of the plasma membrane, wherein a difference in the repair of the plasma membrane relative to the repair of the plasma membrane in a control injured cell is an indication that the candidate compound modulates the repair of the plasma membrane. In some embodiments, a compound that increases the repair of the plasma membrane relative to the repair of the plasma membrane in a cell not contacted with the candidate compound is an enhancer of repair of the plasma membrane. In certain embodiments, a compound that decreases the repair of the plasma membrane relative to the repair of the plasma membrane in a cell not contacted with the candidate compound is an inhibitor of repair of the plasma membrane. In some embodiments, the cell is a normal cell.

According to another aspect of the invention, methods for identifying abnormal cell plasma membrane repair in a cell are provided. The methods include injuring a plasma membrane of a sample cell by contacting the sample cell with a device that exerts force on the sample cell equal to about the weight of the device under gravity, and measuring the repair of the sample cell plasma membrane, wherein a difference in the repair of the sample cell plasma membrane relative to the repair of a plasma membrane in a injured control cell is an indication of abnormal cell plasma membrane repair in the sample cell. In some embodiments, the sample cell is a cell suspected of having a plasma membrane repair-associated disorder. In certain embodiments, the plasma membrane repair-associated disorder is a muscular dystrophy (e.g., Limb-girdle muscular dystrophy type 2B (LGMD2B), Miyoshi myopathy); otoferlin-associated deafness; a metabolic disorder (e.g. rhabdomyositis); calpain mutation-associated disease [e.g. Limb-girdle muscular dystrophy type 2A (LGMD2A)]; caveolin mutation-associated disease [e.g. Limb-girdle muscular dystrophy type 1C (LGMD1C)]; lysosomal storage disorders (e.g. Chediak-Higashi Syndrome); muscle cell trauma, or other cell trauma. In some embodiments, the muscle cell trauma is exercise-associated muscle trauma or injury associated muscle-cell trauma. In some embodiments, the cell is a normal cell. In some embodiments, the sample cell is modified. In certain embodiments, the modification of the sample cell is a genetic modification or a chemical modification. In some embodiments, the sample cell is from a subject having or suspected of having a muscle disorder or disease. In some embodiments, the sample cell is a cell that has been treated with a compound to alter plasma membrane repair. In some embodiments, the sample cell is from a subject that has been or is being treated for a plasma-membrane repair-associated disease or disorder.

According to another aspect of the invention, methods for monitoring the onset, progression, or regression of a plasma-membrane repair-associated disorder in a subject are provided. The methods include obtaining a first cell sample from a subject, injuring the plasma membrane in the first cell sample by contacting the cell sample with a device that exerts force on the cell plasma membrane equal to about the weight of the device under gravity, and measuring the repair of the plasma membrane in the first cell sample, obtaining a second cell sample from the subject at a time subsequent to the time the first cell sample was obtained, injuring a plasma membrane of the second cell sample by contacting the cell sample with the device that exerts force on the cell plasma membrane equal to about the weight of the device under gravity, and measuring the repair of the second cell sample plasma membrane, wherein a difference in the repair of the first cell sample plasma membrane relative to the repair of the second cells sample plasma membrane is an indication of the onset, progression, or regression of the plasma membrane repair-associated disorder in the subject. In some embodiments, the subject is undergoing treatment for a plasma membrane repair-associated disorder. In certain embodiments, the sample cell is from a subject having or suspected of having a muscle disorder or disease. In some embodiments, the plasma membrane repair-associated disorder or disease is a muscular dystrophy (e.g., Limb-girdle muscular dystrophy type 2B (LGMD2B), Miyoshi myopathy); otoferlin-associated deafness; a metabolic disorder (e.g. rhabdomyositis); calpain mutation-associated disease [e.g. Limb-girdle muscular dystrophy type 2A (LGMD2A)]; caveolin mutation-associated disease [e.g. Limb-girdle muscular dystrophy type 1C (LGMD1C)]; lysosomal storage disorders (e.g. Chediak-Higashi Syndrome); muscle cell trauma, or other cell trauma. In certain embodiments, the muscle cell trauma is exercise-associated muscle trauma or injury associated muscle-cell trauma. In some embodiments, the cell is a normal cell. In some embodiments, the cell sample has been treated with a compound to alter plasma membrane repair. In some embodiments the cell sample is from a subject that has been or is being treated for a plasma-membrane repair-associated disease or disorder. In certain embodiments, the cell sample is cultured prior to being injured. In some embodiments, the cell sample includes muscle cells. In some embodiments, the cell sample is in a multi-well plate.

According to yet another aspect of the invention, methods for identifying genetic modifications that modulate cell plasma membrane repair are provided. The methods include injuring a plasma membrane of a genetically modified cell by contacting the cell with a device that exerts force on the cell equal to about the weight of the device under gravity, and measuring the repair of the plasma membrane, wherein a difference in the plasma membrane repair relative to the plasma membrane repair in a control injured cell is an indication that the genetic modification modulates plasma membrane repair. In some embodiments, a genetic modification that increases the repair of the plasma membrane relative to the repair of the plasma membrane in a cell that does not have the genetic modification is an enhancer of plasma membrane repair. In certain embodiments, a genetic modification that decreases the repair of the plasma membrane relative to the repair of the plasma membrane in a cell that does not have the genetic modification is an inhibitor plasma membrane repair. In some embodiments, the genetic modification is the delivery of a nucleic acid into the cell. In some embodiments, the nucleic acid is a cDNA. In some embodiments, the cDNA is a dysferlin cDNA.

In some embodiments of the foregoing aspects of the invention, the cell is a cultured cell. In certain embodiments of the foregoing aspects of the invention, the cell is in a multi-well plate. In certain embodiments of the foregoing aspects of the invention, the cell is a muscle cell. In some embodiments of the foregoing aspects of the invention the cell is a fibroblast. In some embodiments of the foregoing aspects of the invention, the cell is initially contacted with the candidate compound before the time the cell is injured. In certain embodiments of the foregoing aspects of the invention, the cell is initially contacted with the candidate compound during or after the time the cell is injured. In some embodiments of the foregoing aspects of the invention, the device is a floating pin. In some embodiments of the foregoing aspects of the invention, the floating pin is one of a plurality of floating pins. In some embodiments of the foregoing aspects of the invention, the plurality of pins spatially correspond to the wells of a multi-well plate. In certain embodiments of the foregoing aspects of the invention, the plurality of floating pins is a floating pin tool. In some embodiments of the foregoing aspects of the invention, the floating pin tool is robotically operated. In certain embodiments of the foregoing aspects of the invention, the floating pin tool is manually operated. In some embodiments of the foregoing aspects of the invention, repair is measured with a microplate reader. In some embodiments, the step of measuring the repair includes contacting the cell with a toxin and determining cell death and/or cell viability. In certain embodiments of the foregoing aspects of the invention, the toxin is a cell-impermeable toxin. In some embodiments of the foregoing aspects of the invention, the cell-impermeable toxin is Gelonin or Granzyme B. In some embodiments of the foregoing aspects of the invention, the step of measuring the repair includes determining the release of a molecule from the cell. In certain embodiments of the foregoing aspects of the invention, the molecule released from the cell is an enzyme. In some embodiments of the foregoing aspects of the invention, the enzyme is lactate dehydrogenase (LDH) or β-hexosaminidase. In some embodiments of the foregoing aspects of the invention, the step of measuring the repair includes determining the entry into the cell of a molecule that does not enter the cell in the absence of a plasma membrane injury. In some embodiments of the foregoing aspects of the invention, the molecule that does not enter the cell in the absence of a plasma membrane injury is dextran. In certain embodiments of the foregoing aspects of the invention, the molecule that does not enter the cell in the absence of a plasma membrane injury is detectably labeled. In some embodiments of the foregoing aspects of the invention, the detectable label is a fluorescent label. In some embodiments, the fluorescently labeled molecule is dextran. In certain embodiments of the foregoing aspects of the invention, the step of measuring the repair includes determining a marker that is selectively expressed by a cell with a repaired cell plasma membrane. In some embodiments of the foregoing aspects of the invention, the marker that is selectively expressed by the cell is Lamp1.

According to yet another aspect of the invention, kits for determining plasma membrane repair in a cell are provided. The kits include one or a plurality of floating pins, and instructions for use of the pin to injure the plasma membrane of a cell and for determining the rate and/or amount of plasma membrane repair in the injured cell. In some embodiments, the plurality of pins spatially correspond to the wells of a multi-well plate. In certain embodiments, the plurality of floating pins is a floating pin tool. In some embodiments, the floating pin tool is robotically operated. In some embodiments, the floating pin tool is manually operated. In some embodiments, repair is measured with a microplate reader. In certain embodiments, the kit also includes a container containing cells. In some embodiments, the kit also includes an exogenous marker molecule. In some embodiments, the marker molecule is dextran. In certain embodiments, the dextran is detectably labeled dextran. In some embodiments, the marker molecule is a plasma membrane impermeable toxin. In some embodiments, the impermeable toxin is Gelonin or Granzyme B. In some embodiments, the kit also includes a container containing solutions for determining the presence of Lamp1, lactate dehydrogenase (LDH) or β-hexosaminidase. In some embodiments, the kit also includes a multi-well plate. In some embodiments, the geometry of the wells of the multi-well plate spatially correspond to the plurality of pins.

These and other aspects of the invention will be described in further detail in connection with the detailed description of the invention.

### BRIEF DESCRIPTION OF THE FIGURES

FIG. 1 is a schematic diagram of a flowchart of the membrane repair assay using a floating pin tool.

### DETAILED DESCRIPTION OF THE INVENTION

We have discovered methods for assessing repair of cell plasma membranes. The methods allow the rapid and reproducible evaluation of the status of plasma membrane repair in a cell sample and/or simultaneously in a plurality of cell samples, thereby allowing high-throughput analysis of plasma membrane repair. The methods of the invention can be used to screen for therapeutic compounds that modulate plasma membrane repair. Thus, cells known to have abnormal (e.g. reduced) ability for cell plasma membrane repair may be tested using the methods of the invention to identify candidate compounds that alter cell plasma membrane repair. For example, the methods of the invention can be used to identify compounds that increase the ability of a cell to repair its plasma membrane and/or compounds that reduce the time necessary for plasma membrane repair in a cell.

In some embodiments, the methods of the invention may be used to determine plasma membrane repair in normal cells and to identify compounds or treatment methods that improve the rate and/or amount of plasma membrane repair in injured normal cells. For example, a normal muscle cell may be tested using the methods of the invention that include injuring a normal muscles cell sample, contacting the injured sample with a candidate plasma membrane repair modulating compound, and determining whether the compound increases the rate of repair of the plasma cell membrane. An increase in the rate of plasma membrane repair indicates that the compound may be useful to decrease healing time for normal cells that undergo injury in vivo, e.g. muscle injury.

The methods of the invention are also useful for the assessment of diseases and/or disorders that are associated with plasma membrane repair in cells, tissues, and subjects. Examples of diseases and disorders associated with plasma membrane repair include, but are not limited, to muscular dystrophies and cell damage from injury or trauma. Cells (e.g. muscle cells) may be damaged through use or injury, and cell plasma membrane repair may be a component of the cell's healing process. The methods of the invention can also be used to determine the ability of a cell to repair its plasma membrane and/or to determine whether a cell of interest has a normal or abnormal ability to repair its plasma membrane.

As used herein the phrase “plasma membrane repair-associated disorder” means a disorder in which there is a reduction in the ability of a cell to repair a plasma membrane injury and also includes disorders or conditions in which there is plasma membrane injury in cells, tissues and/or subjects. Examples of plasma membrane repair-associated disorders include, but are not limited to: muscular dystrophy (e.g., Limb-girdle muscular dystrophy type 2B (LGMD2B), Miyoshi myopathy); otoferlin-associated deafness; a metabolic disorder (e.g. rhabdomyositis); calpain mutation-associated disease [e.g. Limb-girdle muscular dystrophy type 2A (LGMD2A)]; caveolin mutation-associated disease [e.g. Limb-girdle muscular dystrophy type 1C (LGMD1C)]; lysosomal storage disorders (e.g. Chediak-Higashi Syndrome); muscle cell trauma, or other cell trauma. In some embodiments of the invention, muscle cell trauma is exercise-associated muscle cell trauma or injury-associated muscle cell trauma.

As used herein the term “repair” means to reduce the size of an injury to a plasma membrane. In some embodiments, a repair is a complete elimination of the injury. In other embodiments a repair can be less than a complete elimination of the injury, and thus may be a reduction in extent and/or size of the injury that is less than 100%. As used herein, plasma membrane repair is any statistically significant reduction in a plasma membrane injury, and includes a reduction of the size of an injury from about 1%, 5%, 10%, 20%, 30%, 40%, 50%, 60%, 70%, 80%, 90%, through 100% (including all percentages in between), with 100% being the complete elimination of the injury. As used herein, the term “repair status” means the state or amount of repair of a plasma membrane. Thus, the repair status reflects the level of repair that is present in a cell membrane and can be assessed by determining the cell's recovery from plasma membrane injury.

In addition to the size of injury, another aspect of repair that can be assessed using the methods of the invention is the rate or speed of repair of an injured plasma membrane. The rate of plasma membrane repair can be assessed using the methods of the invention by determining the amount of time it takes for cell plasma membrane repair. For example, the amount of time it takes for a cell to reach a certain percentage of repair can be determined and compared to a test or control time for repair. In some embodiments, the length of time for about 90% to 100% plasma membrane repair in a normal cell ranges from about 2, 3, 4, 5, 6, to about 7 seconds. In contrast, the length of time for about 90% to 100% plasma membrane repair in cells with plasma membrane repair-associated disorders may range from about 9, 10, 11, 12, 13, 14, 15, 16, 17, 18, 19, 20, 21, 22, 23, 24, 25, 30, 40, 50, 60, 90, 100, or more seconds (including all times in between). In addition, the cell plasma membrane repair rate can be determined by assessing cell plasma membrane repair status of a membrane over time. Cells similarly prepared and injured can be assessed for repair status at two or more time points following injury as an assessment of change in rate of plasma membrane repair and the amount of time it takes to reach a certain percentage of plasma membrane repair. The methods of the invention are also useful to determine the effect of candidate modulators of plasma membrane repair on the rate of plasma membrane repair. For example, methods of the invention include, in some aspects, contacting the cells with candidate modulators of cell plasma membrane repair and determining the ability of the modulators to alter the rate of plasma membrane repair.

The methods of the invention include the use of tools to physically injure a cell plasma membrane. The methods include physically injuring a plasma membrane of a cell by contacting the cell with a device that exerts force on the cell plasma membrane about equal to the weight of the device under gravity. As used herein, the term “device” is a tool or implement that when contacted with a cell surface area the force of the contact is approximately equal to the gravitational force on the tool or implement. In some embodiments, a device is a floating pin and the invention includes the use of a floating pin for injuring a cell plasma membrane.

Generally, a floating pin is a cylinder-shaped (e.g. elongated) pin or rod that is not fixed in a base, but rather can “float” or move upward from its point of contact when the tip of the floating pin contacts a surface. As a result, the downward force exerted by the pin onto the surface contacted by the tip of the floating pin is equal to about the weight of the pin under gravity. Floating pins are known in the art and those of ordinary skill will recognize that one or more floating pins may be mounted in a floating pin tool or floating pin replicator. The one or more floating pins in a floating pin tool or replicator can be simultaneously operated (e.g. moved) by manual or robotic means. One of ordinary skill will recognize that some additional forces such as the buoyancy of the pin in the culture fluid and the compression force of the culture fluid on the cell as the pin descends may be forces that modulate the gravitational force on the pin, but are so small as to not affect the floating nature of the pin. Those of ordinary skill will recognize that these additional forces will be the same in all samples when the floating pins are substantially similar and are used under substantially similar conditions to produce plasma cell membrane injury in the samples.

Various models of floating pins and floating pin tools are commercially available [see for example: V&P Scientific, Inc. (San Diego, Calif. www.vp-scientific.com/)]. Those of ordinary skill in the art will recognize that numerous alternative designs of floating pins and floating pin tools are available and can be used in the methods of the invention. The design of a floating pin tool is such that a plurality (two or more) of floating pins is arrayed in a holder such that each pin retains its independent, vertical, gravity-based floating movement ability, but the pins can be applied to a surface or a plurality of surfaces (e.g., wells in a multi-well plate) simultaneously. Thus, using a floating pin tool, a plurality of cell samples may be simultaneously contacted with a plurality of floating pins that are arrayed in the floating pin tool and the force with which each pin contacts a cell surface area will be about equal to the force of gravity on that single pin.

Floating pins of the invention can be arrayed in numerous arrangements as long as they can be used in the methods of the invention to produce an injury in a cell plasma membrane that results from the force of gravity on a pin, substantially independent of other forces. One of ordinary skill in the art will recognize that the term “floating pin tool” encompasses floating pin tools with numerous different arrangements of pins and that a floating pin tool may be constructed in alternate ways than those described herein. Alternative pin and pin tool designs and arrangements are encompassed by the methods of the invention as long as they provide a floating pin or a plurality of floating pins that can be used for cell plasma membrane injury as described herein.

In some embodiments of the invention, the floating pin is a component of a pin tool that is robotically operated to injure the cell surface area. In other embodiments, the pin is manually operated to injure the cell surface area. Regardless of whether the floating pin is operated manually or robotically, the resulting contact between the pin and a cell plasma membrane results from the force of gravity on a pin, substantially independent of other forces.

In some embodiments of the invention, the pin tool includes a plurality of pins that spatially correspond to the wells of a multi-well plate and in some embodiments, the assay endpoint (which is a measure of membrane repair status) is measured with a microplate reader. As used herein the term “spatially corresponds” means to match up spatially. For example the plurality of floating pins are located in an array that matches the array of wells in the multi well plate and can simultaneously enter the plurality of wells.

There can be wide variation in the geometry and construction of pins useful in the methods of the invention. Pins may be hollow or solid and may be notched, pointed, or flat at their plasma membrane-contacting end. A pin useful in the methods of the invention may be made of one or more materials including, but not limited to, metal (e.g. stainless steel), glass, plastic, resin, polycarbonate, etc. In some embodiments, a pin used in the methods of the invention can be coated with a hydrophobic or other type of coating and in other embodiments the pin can be uncoated. The cross-sectional shape of the pin may be round, square, rectangular, oval, or other shape as long as the pin can be used to injure a cell plasma membrane according to the methods of the invention. Features such as pin weight, pin diameter, pin tube holder diameter, buoyancy, pin material, pin surface geometry, growth medium density, and the relationship between the pin diameter and the diameter of the cell growth surface, etc., are parameters that can be set and/or adjusted by one of ordinary skill in the art to optimize the methods of the invention.

The methods of the invention include, in part, placing cells into a vessel (e.g., a well of a 96-well plate), culturing the cells in the vessel, and contacting the cells with a floating pin one or more times to injure the cell plasma membrane. Examples of vessels that are useful in the methods of the invention, though not intended to be limiting, are dishes, vials, tubes, and wells. In some embodiments, a vessel may be a well of a multi-well plate. As used herein, the term “culture” means to grow cells in a vessel. When grown in culture, the cells attach to the surface of the vessel forming a layer of cells across the growth surface of the vessel. In some embodiments, the layer of cells is a confluent layer. As used herein, the term “cell surface area” is the surface area of cultured cells in a vessel. An example of a cell surface area in a vessel for use in a cell plasma membrane repair assay of the invention, is the area of a well in a 96-well plate well that is covered by cultured cells.

The methods of the invention, in part, include contacting a cell with a candidate plasma membrane repair-modulating compound before, during, or after the cell is physically injured with a device (e.g. floating pin). The plasma cell membrane of the cell can be injured by contacting the cell plasma membrane with a device (e.g. the floating pin) 1, 2, 3, 4, 5, 6, 7, 8, 9, 10, or more times. One of ordinary skill in the art will recognize that the number of contacts between the device and the cell plasma membrane can be optimized based on assay features such as the type of cell, the extent of injury desired, the culture conditions, etc. In addition, features of the device (e.g. the floating pin) may also be used to optimize the number of contacts between the device and the cell to be injured. These features include, size of the region of the device that contacts the cell, geometry of the region of the device that contacts the cell, etc. For example, a different number of contacts may be optimal to injure a cell plasma membrane when using a pin with a pointed end that contacts a cell surface area than would be optimal when using a pin with a flat end that contacts a larger cell surface area. These and other device and assay parameters will be understood by those of ordinary skill to be considered for optimizing the methods of the invention with respect to the number of contacts between a device and a cell to cause cell plasma membrane injury.

The methods of the invention include insertion of a floating pin into a vessel that contains cells to be injured and contact of the floating pin one or more times with the cultured cells in the vessel. The fit of the pin in the vessel and the percentage of the cell surface area contacted by the pin can vary depending on the shape and size of the pin and the cell surface area. The percentage of contact area between the floating pin and the cell surface area can be standardized from vessel to vessel and experiment to experiment. In some embodiments, the size of the surface of the floating pin that contacts the cell surface area can be a percentage of the size of the cell surface area. For example, the surface area of the pin that contacts the cells can be up to 5%, 10%, 15%, 20%, 25%, 30%, 35%, 40%, 45%, 50%, 55%, 60%, 65%, 70%, 75%, 80%, 85%, 90%, 95% or more (including all percentages in between) of the size of the cell surface area in the vessel. In some embodiments, the surface area of the pin that contacts the cells can be between about 20% and about 70% of the size of the cell surface area in the vessel. In preferred embodiments, the surface area of the pin that contacts the cells can be between about 40% and about 60% of the size of the cell surface area in the vessel. In particularly preferred embodiments, the surface area of the pin that contacts the cells can be between about 45% and about 55% of the size of the cell surface area in the vessel.

For screening cultured cells in more than one vessel simultaneously, e.g. high-throughput screening, an approximately equal number of cells can be added to each vessel and cultured. For example, approximately 5, 10, 20, 30, 40, 50, 100, 200, 300, 400, 500, 1000, 1500, 2000, 2500, 3000, 3500, 4000, 4500, 5000, 5500, 6000, 6500, 7000, 7500, 8000 or more (including all integers in between) cells can be added to each of one or more wells of a 96-well plate. It will be clear to one of ordinary skill in the art that the number of cells placed in each vessel (e.g. well) will depend on factors such as cell size, growth characteristics (e.g. optimal density for growth), vessel surface area available, etc. In some embodiments of the invention, the cells are a confluent culture of cells. As used herein, use of a confluent culture permits use of substantially identical numbers of cells. For example, a confluent culture that is seeded and grown in a vessel will contain a substantially equal number of cells as a confluent culture, cultured under identical conditions in a substantially similar vessel.

Having a substantially consistent numbers of cells per vessel is an important feature in the methods of the invention relating to high-throughput screening. Substantial consistency in the number of cells per vessel is useful for comparing cell plasma membrane repair status between vessels and/or between experiments. For example, in some embodiments, approximately 5000 muscle cells can be placed and cultured in each of a plurality of wells of a 96-well plate, thus allowing interwell comparisons of cell plasma membrane repair under identical or varying conditions (e.g. control versus contact with a candidate repair-modulating agent). It will be understood that substantially identical numbers of cells per vessel are not required to practice the invention and that assay endpoint values can be extrapolated based on the approximate number of cells per vessel allowing relative comparisons of repair status results from assays of cells in vessels that do not have substantially consistent numbers of cells per vessel. Additional examples of possible comparisons between vessels and/or samples include, but are not limited to, comparison of repair status levels per approximate or average number of cells and comparison of repair status levels per area (e.g., surface area) of cells tested.

According to the methods of the invention, an approximately equal number of a type of cells plated into substantially identical vessels (e.g. wells of a 96-well plate) and cultured under identical conditions will have substantially equal cell surface areas. Contact of the cell surface areas with substantially identical floating pins allows equivalent cell plasma membrane injuries to be made to the cell surface area in each well, allowing screening of multiple cell surface areas in parallel with high-throughput assays. In one embodiment, equal numbers of cells are plated in wells of a 96-well plate and a floating pin tool with a multi-pin conformation that spatially corresponds to wells of the 96-well plate can be used to simultaneously and substantially identically injure the plasma cell membranes of the cells in culture in the multiple wells of the plate.

Cells from a number of different sources can be used in the methods and kits of the invention. Cells may be cells obtained from a subject for whom a diagnosis is desirable, genetically modified cells, cells from a normal subject, cell lines in culture, etc. The methods of the invention also allow diagnostic assessments of cell samples that can be used for initial diagnosis and/or for monitoring treatment strategies in cells and subjects. Cells useful in the methods of the invention include human as well as other mammalian cells such as cells from non-human primates, cats, dogs, sheep, pigs, horses, cows, rodents such as mice, hamsters, and rats. In addition, cells from non mammalian organisms may also be used in the methods of the invention. Cells from avian species, fish, insect, amphibians, reptiles etc., may be used in the methods of the invention. For example, Drosophila, Xenopus, nematode, and zebrafish etc., cells may be used in the methods of the invention. Human and non-human cells useful in the methods of the invention include cells made or selected for use as cell models for plasma membrane repair-associated diseases and/or injuries.

Numerous types of cells are useful in the methods of the invention. Examples of cell types that can be used in the methods of the invention, include but are not limited to: muscle cells (e.g. mouse C2C12 cells, rat PC12 cells, dysferlin-deficient cell lines, etc.) and fibroblasts (e.g. mouse 3T3 cells, Beige J fibroblasts that are a model for Chediak-Higashi disease, etc.), embryonic and other stem cells, neurons, glial cells, etc. In addition, one of ordinary skill in the art will recognize that cells that exhibit a patch-fusion membrane repair response can also be used in the methods of the invention. Examples of such cells, though not intended to be limiting include NRK cells, L6E9 cells, CHO cells, RGM1 cells, BAE cells, and HUSMC cells. Cells that exhibit normal plasma cell membrane repair and cells in which plasma cell membrane repair is abnormal can be used in the methods provided herein. In some embodiments, muscle cells in culture may be further contacted with fusion medium for the differentiation to multinucleated myotubes. Those of ordinary skill in the art will recognize that the methods of the invention can also be used to determine cell plasma membrane repair status in alternative cell types, in addition to those specifically provided herein.

The invention includes assays to identify compounds that modulate the cell plasma membrane repair (e.g. rate or level of repair) and also includes, in some aspects methods to identify cells, tissues, and/or subjects that have a cell plasma membrane repair-associated disorder. Thus the methods and kits of the invention relate to plasma membrane injury repair. In some embodiments, plasma membrane repair assay endpoint results from a test cell are compared to control plasma membrane repair assay endpoint results. In some embodiments, the repair of a plasma cell membrane contacted with a candidate compound is compared to the repair of the plasma membrane in a physically injured cell not contacted with the compound (a control) as a determination of whether the candidate compound modulates the repair of the plasma membrane.

Levels and/or rates of cell plasma membrane repair are preferentially compared to controls. The control may be a predetermined value, which can take a variety of forms. It can be a single value, such as a median or mean. A control value can be established based upon comparative groups (e.g. comparative cell types), such as in cells having normal amounts and/or rates of cell plasma membrane repair. It will be understood that the “normal” amount and/or rate of cell plasma membrane repair may be the amount or rate of repair determined in a cell culture that is not contacted with a candidate repair-modulating compound. This “normal” value can serve as a control value for a substantially similar cell culture that is contacted with a candidate repair-modulating compound. In some embodiments, a control may be the level and/or rate of cell plasma membrane repair from a culture of cells that are free of a disease or disorder present in the test sample. A control may also be the level and/or rate of cell plasma membrane repair in a wild-type cell culture. Once a compound or agent that modulates rate and/or level of cell plasma membrane repair has been identified, additional testing can be done to determine the effect of the compound on rate and/or level of cell plasma membrane repair in cells and tissues.

Cells for plasma membrane repair testing may be cells from subjects and/or cultured cells known to have, or suspected of having a particular disease [e.g., muscular dystrophy (e.g., Limb-girdle muscular dystrophy type 2B (LGMD2B), Miyoshi myopathy); otoferlin associated deafness; a metabolic disorder (e.g. rhabdomyositis); calpain mutation-associated disease [e.g. Limb-girdle muscular dystrophy type 2A (LGMD2A)]; caveolin mutation-associated disease [e.g. Limb-girdle muscular dystrophy type 1C (LGMD1C)]; lysosomal storage disorders (e.g. Chediak-Higashi Syndrome); muscle cell trauma, or other cell trauma]. In some embodiments of the invention, muscle cell trauma is exercise-associated muscle cell trauma. Additionally, cells from subjects with condition or symptoms, and cells from groups without the disease, condition or symptoms can be used in the injury repair assessment methods of the invention. Another comparative cell type would be cells from subjects with a family history of a disease or condition and a group without such a family history. Another group of cells that can be used in the methods of the invention are cultured cells that have been treated or cells from a subject who has been treated for a cell plasma membrane repair-associated disorder or disease. Additionally, the methods of the invention are useful to measure a first sample from a subject cell source and a subsequent sample from the subject or cell source to assess changes in the cell plasma membrane repair status for the subject, that may result from onset, progression, regression or treatment of the cell plasma membrane repair-associated disorder or disease.

A predetermined control value of course, will depend upon the particular population of cells selected. For example, an apparently healthy cell population will have a different ‘normal’ range of cell plasma membrane repair rates and levels than will a population that is known to have a cell plasma membrane repair-associated disease or condition. Accordingly, the predetermined value selected may take into account the category in which a cell type falls. Appropriate ranges and categories can be selected with no more than routine experimentation by those of ordinary skill in the art. By abnormal rates or levels it is meant abnormal (high or low) relative to a selected control. It will also be understood that the controls for use in the invention may be, in addition to predetermined values, samples of materials tested in parallel with the experimental materials. Examples include samples from control cells or control samples (e.g., generated through manufacture) to be tested in parallel with the experimental samples.

Methods to assess function of the plasma membrane can be used as a measure of the ability of a cell to repair its plasma membrane. As used herein the term “functional” repair means sufficient to reduce or eliminate cell death or significant loss of function in the test cell. In addition to functional repair, the amount of time it takes for a cell to repair its plasma membrane can be determined and can serve as a measure of the cell's repair ability and rate of repair. Differences in these repair parameters, compared to the same parameters in a control cell can be used as an indication that the test cell has abnormal cell plasma membrane repair. As used herein, the term “abnormal” means different from the rate or level of plasma membrane repair in a control cell. In some embodiments, an abnormal ability of a test cell to repair its plasma membrane is a reduced ability to repair as compared to a control cell. In other embodiments, an abnormal ability of a cell to repair its plasma membrane is an increased ability of the test cell to repair compared to a control cell's ability to repair its plasma membrane. Thus, a determination of functional repair in a test cell can be compared to functional repair in a control cell as a measure of the functional repair ability of the test cell. Similarly, a determination of the time it takes for plasma membrane repair (e.g. the rate of repair) in a test cell can be compared to the time necessary for repair in a control cell as a measure of the repair ability of the test cell.

With the methods of the invention, the rate of repair can be determined. Methods to determine rate may include, but are not limited to comparing the rate of repair (e.g. a change in percentage of repair over time) for a test sample with a control sample. If the rate is reduced in the test cell, it demonstrates that the cell has a plasma cell membrane-repair associated disorder or disease. In some embodiments, a plasma cell membrane repair-associated disorder or disease can be identified by a determination that the cell plasma membrane repair is slower that repair in a normal control cell. In some embodiments, a plasma cell membrane repair-associated disorder or disease can be identified by a determination that the cell plasma membrane repair is slower that repair in a normal control cell. The effect of a candidate repair modulating compound can be determined by contacting a cell plasma membrane-injured cell with the candidate molecule and/or compound and comparing the rate of repair in the contacted cell with the rate of repair in a control cell not contacted with the candidate. The comparison can demonstrate a modulatory effect of the candidate molecule and/or compound (e.g. either an increase or decrease) in plasma cell membrane repair.

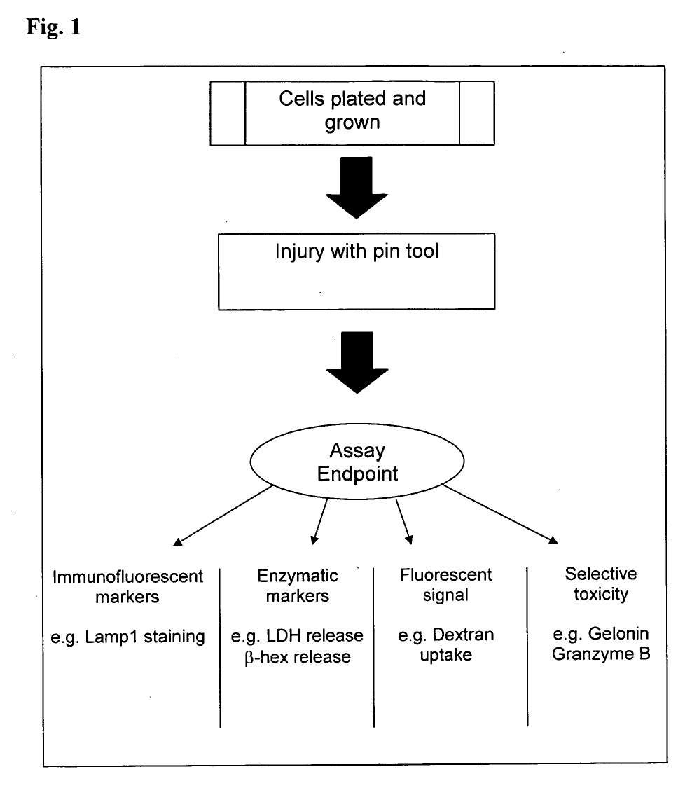

The methods of the invention include the use and assessment of one or more assay endpoints that indicate the extent and/or rate of plasma membrane repair. As used herein, the term “assay endpoint” means determination of a marker that is a measure of plasma membrane repair. FIG. 1 illustrates several possible assay endpoints that are useful for determining plasma membrane repair in a cell, tissue, and/or subject. Plasma membrane repair can be evaluated by assessing plasma membrane function. Membrane injury may result in a reduction of the normal “barrier” function of the plasma membrane. Thus, a plasma membrane injury may be accompanied by an increase permeability of a cell's plasma membrane to intracellular and/or extracellular molecules. An increase in the release of intracellular molecules or the entry of extracellular molecules into a cell is an assay endpoint that can be monitored as a measure of plasma membrane injury. In some embodiments, an exogenous molecule can be put into the extracellular environment of a cell as a marker for increased cell membrane permeability and measured as a determination of the repair status of the membrane. For example, detectibly labeled molecules that are too large to enter a cell through an intact plasma membrane (e.g. dextrans) can be placed into the extracellular environment and monitored. In some embodiments, the extracellular environment may be washed following placement of the marker in the extracellular environment to reduce extracellular background and enhance signal-to-noise ratio in the determination of the marker in the cell. The entry of a marker such as dextran into a cell can be used as an indication of plasma membrane injury and repair status of the cell. In some embodiments, dextran is the molecule that enters as a result of injury and the level of dextran in the cell can be assessed as an assay endpoint. In some embodiments, the dextran is detectably labeled dextran. An example, although not intended to be limiting, of a detectable label that can be used to monitor dextran location using the methods provided is fluorescein. It will be understood by those in the art that other membrane impermeant molecules can be assessed as markers for plasma membrane repair using the methods of the invention.

Methods of the invention that utilize the determination of extracellular (e.g. exogenous) molecules inside an cell following injury include, but are not limited to, contacting an injured cell with a cell-impermeable toxin and determining the effect of the toxin on the cell. If an injured cell is contacted with a cell-impermeable toxin and the cell is affected by the toxin (e.g. is damaged or dies), it indicates that the cell has a plasma membrane injury and complete plasma membrane repair has not occurred. In contrast, an injured cell that is similarly contacted with the toxin but is not affected by the toxin, indicates plasma membrane repair has occurred in the cell. Examples of toxins that can be used in the methods of the invention to assess plasma membrane repair status include, but are not limited to: cell impermeable toxins such as Granzyme B and Gelonin. Those of skill in the art will recognize that additional cell-impermeable toxins can be used in the methods of the invention. Thus, in some embodiments of the invention, the assay endpoint is the measure of cell toxicity (e.g. cell death) following cell plasma membrane injury and contact with a cell-impermeable toxin.

Another method of the invention for assessing repair status of a plasma membrane is the monitoring of molecules release associated with membrane repair. In some embodiments, the molecules released are enzymes. Membrane repair includes the fusion of intracellular vesicles systems with the cell surface membrane. A result of this process is the release of the contents of the vesicles into the extracellular environment of the cell. The invention includes methods of measuring the release of enzymes to assess repair status and rate of repair. Examples of molecules released by vesicle fusion include, but are not limited to, lactate dehydrogenase (LDH) and β-hexosaminidase. Thus, in some embodiments of the invention, the assay endpoint is the measure of LDH, β-hexosaminidase or other molecule, whose release to the extracellular environment is associated with plasma membrane repair, and is a marker for plasma membrane repair status.

In addition to altered movement of molecules across the plasma membrane in injured cells, the repair of a cell's plasma membrane may be accompanied by selective expression of one or more proteins associated with the plasma membrane repair process. Selectively expressed proteins can be assessed as an indication of the repair status of a cell. Thus, in some embodiments, the invention includes methods of assessing markers on the surface of cells that have been injured as a measure of plasma membrane repair. For example, lysosomal-associated membrane protein 1 (Lamp1) is selectively expressed on the surface of repaired muscle cells. Methods of the invention include quantification of Lamp1 expression in a cell that has been injured, as an indication of plasma membrane repair in the cell. Additional repair-specific proteins and other markers can also be examined as a measure of plasma cell membrane repair. Thus, in some embodiments of the invention, the assay endpoint is the measure of expression of a membrane repair-associated molecule. In some embodiments, the membrane repair-associated molecule is Lamp1.

Molecules and compounds that can be monitored using the methods of the invention to assess plasma membrane repair are referred to herein as marker molecules. A marker molecule is a molecule whose level or location (e.g. intracellular or extracellular location) can be evaluated as an indication of the repair status of a plasma membrane. Methods to evaluate the presence and/or level of a marker molecule may include the use of detectible labels. Marker molecules can be coupled to specific labeling agents, for imaging of cells and tissues according to standard coupling procedures. A wide variety of detectable labels can be used, such as those that provide direct detection (e.g., a radioactive label, a fluorophore, [e.g. Green Fluorescent Protein (GFP), Red Fluorescent Protein (RFP), etc.], a chromophore, an optical or electron dense label, etc.) or indirect detection (e.g., an epitope tag such as the FLAG epitope, enzyme tag such as horseradish peroxidase, etc.). A variety of methods may be used to detect the label, depending on the nature of the label and other assay components. Labels may be directly detected through optical or electron density, radioactive emissions, nonradiative energy transfers, etc. or indirectly detected with antibody conjugates, strepavidin-biotin conjugates, etc. Methods for detecting the labels are well known in the art. In some important embodiments of the invention, assay endpoint determinations can be done using multi-well plate readers (e.g. microplate reader) to assess the amount and/or location of a marker molecule. Additional methods for detecting labels are well known in the art and can be used in the methods of the invention.

Marker molecules of the invention may be endogenous molecules or exogenous molecules as long as they are useful for determining plasma membrane repair status. In some embodiments, plasma membrane repair status can be determined using methods that introduce exogenous agents into the environment of the cell coupled with assessment of cell viability and/or marker amount or location. A number of assay endpoints are described herein. These include assessment of endogenous or exogenous compounds and/or molecules that are affected by plasma membrane injury and/or repair.

As used herein, the term “exogenous” agent or marker molecule means a compound that does not naturally occur in a test cell or its environment but is added to the environment as a means to measure plasma membrane repair. Examples of exogenous agents that can be used in the methods of the invention include, but are not limited to: dextran (e.g. detectably labeled dextran), toxins (e.g. membrane impermeant toxins such as Gelonin, and Granzyme B), etc. In some embodiments, the Gelonin can be an extract from Gelonin Multiforum seeds or a recombinant protein.

As used herein the term “endogenous” agent or marker molecule, whose level or location can be determined as an assay endpoint in the methods of the invention are molecules that naturally occur in or on a cell or in the cell's environment under injury and/or repair conditions. As used herein the term “endogenous” includes materials that may leak from a cell and/or material specifically synthesized and/or secreted as part of the injury or repair process. Examples of endogenous agents or marker molecules that are useful in the methods of the invention include, but are not limited to Lamp1, lactate dehydrogenase (LDH) and β-hexosaminidase. Those of skill in the art will recognize that additional endogenous and exogenous marker molecules can be used in the methods of the invention to assess plasma membrane repair status.

Screening Methods for Plasma Membrane Repair-Modulating Molecules

The invention, in part, includes methods for screening for and identifying compounds that modulate cell plasma membrane repair. As used herein, the term “modulate” means to change, which in some embodiments means to “enhance” or “increase” and in other embodiments, means to “inhibit” or “reduce”. As used herein, the term “compound” can be a molecule or combined molecules (e.g. a complex of two or more molecules in association with each other). A cell plasma membrane repair-modulating compound is a compound that changes (e.g. enhances or inhibits) cell plasma membrane repair. The methods of the invention include cell-based (in vitro) assays of various kinds. Cell-based assays may include mechanically injuring a cell plasma membrane and determining the rate and/or level of the cell relative to a control rate and/or level of repair. In some embodiments, the methods of the invention include injuring the plasma membrane of a cell and contacting the injured cell with compounds that are candidate modulators of cell plasma membrane repair. Candidate plasma membrane repair-modulating compounds can be screened for modulating (enhancing or inhibiting) cell plasma membrane repair using the assays and assay endpoints described herein (e.g., in the Example section). It is understood that any mechanism of action described herein for the cell plasma membrane repair-modulating compounds is not intended to be limiting, and the scope of the invention is not bound by any such mechanistic descriptions provided herein.

Generally, the screening methods involve assaying for compounds that modulate (enhance or inhibit) the level or rate of cell plasma membrane repair, by assessing the effect of the compound on the rate or level of plasma membrane repair as described herein. The methods of the invention may be used to assess the effect of a candidate repair-modulatory compound on the level or rate of cell plasma membrane repair. These methods include injuring a cell plasma membrane, contacting the injured cell with a candidate compound, and assessing the candidate compound's effect using an assay endpoint, such as those described herein. Examples of assay endpoints that are useful in the cell plasma membrane repair determination methods of the invention include, but are not limited to: determination of Lamp1, determination of LDH release, determination of β-hex release, determination of dextran uptake, determination of selective toxicity (e.g. with Gelonin or Granzyme B). Those of ordinary skill in the art will recognize that additional assay endpoints are also possible in conjunction with the methods of the invention as a determination of the level and/or rate of cell plasma membrane repair.

Assays for identifying plasma membrane repair-modulating molecules and compounds can be used in accordance with this aspect of the invention, including, the high-throughput assays described herein. An example of such an assay that is useful to test candidate cell plasma membrane repair-modulating molecules and/or compounds is provided in the Examples section. In such assays, the assay mixture includes a candidate cell plasma membrane repair-modulating compound. Typically, a plurality of assay mixtures is run in parallel with different agent concentrations to obtain a different response to the various concentrations. Typically, one of these concentrations serves as a negative control, i.e., at zero concentration of the candidate compound or at a concentration of the compound below the limits of assay detection. Immunoassays and fluorescent assays may be used to determine the assay endpoint results according to the invention including sandwich-type assays, competitive binding assays, one-step direct tests and two-step tests such as routinely practiced by those of ordinary skill in the art. In some embodiments, the assay endpoint (which is a measure of membrane repair status) is measured with a microplate reader.

The candidate cell plasma membrane repair-modulating molecules used in the assays of the invention can be natural or synthetic compounds, such as those in small molecule libraries of compounds (including compounds derived by combinatorial chemistry). Natural product libraries also can be screened using such methods, as can selected libraries of compounds known to exert pharmacological effects, such as libraries of FDA-approved drugs. Compounds identified by the assays can be used in therapeutic methods of the invention described below.

Candidate cell plasma membrane repair-modulating molecules of the invention encompass numerous chemical classes, although typically they are organic compounds. In some embodiments, the candidate pharmacological agents are small organic compounds, i.e., those having a molecular weight of more than 50 yet less than about 2500, preferably less than about 1000 and, more preferably, less than about 500. Candidate agents include functional chemical groups necessary for structural interactions with proteins and/or nucleic acid molecules, and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid molecule, the agent typically is a DNA or RNA molecule, although modified nucleic acid molecules as defined herein are also contemplated.

Candidate cell plasma membrane repair-modulating molecules of the invention are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents can be tested and further may be subjected to directed or random chemical modifications such as acylation, alkylation, esterification, amidification, etc. to produce structural analogs of the agents.

A variety of other reagents also can be included in the assay mixtures of the invention. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

In general, the mixture of the foregoing assay materials is incubated under conditions whereby, but for the presence of the candidate pharmacological agent, a control level or rate of cell plasma membrane repair will occur. Some repair modulating molecules or compounds will increase the rate or level of cell plasma membrane repair and some will decrease the rate of level of cell plasma membrane repair, as evidenced by the determination of an assay endpoint such as those described herein. It will be understood that reduction may mean reduction to zero or may mean reduction to a rate or amount below a normal level, a previous level, or a control level. An increase may be an increase to a rate or amount above a normal level, a previous level, or a control level.

Changes in relative or absolute rates and/or amounts of cell plasma membrane repair greater than 0.1% when contacted with a candidate compound in an assay of the invention may indicate a compound that is effective for the prevention and/or treatment of a cell plasma membrane repair-associated disease or disorder. As will be understood by those of ordinary skill in the art, some compounds that modulate cell plasma membrane repair will increase the rate and/or amount of repair and other compounds that modulate cell plasma membrane repair will decrease the rate and/or amount of repair. Preferably, the change in rate and/or amount of cell plasma membrane repair, which indicates a compound is effective, is greater than 0.2%, greater than 0.5%, greater than 1.0%, 2.0%, 3.0%, 4.0%, 5.0%, 7.0%, 10%, 15%, 20%, 25%, 30%, 40%, 50%, 60% 70%, 80%, 90%, 100% or more. As described above, an increase in rate and/or amount of cell plasma membrane repair in an assay of the invention, indicates a compound that may be useful to prevent and/or treat a cell plasma membrane repair-associated disease or disorder.

The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high-throughput screening, and typically are between 1 minute and 10 hours.

As mentioned above, it is possible to determine the efficacy of a candidate compound to modulate cell plasma membrane repair by monitoring changes in the absolute or relative rate and/or amount of the cell plasma membrane repair in the absence and/or presence of a candidate compound. For example, an increase in the rate or amount of cell plasma membrane repair indicates that a candidate compound enhances cell plasma membrane repair in the cell. Similarly, a decrease in the rate or amount of cell plasma membrane repair indicates that a candidate compound reduces cell plasma membrane repair in the cell.

The rate and/or amount of cell plasma membrane repair in a cell contacted with the candidate compound, as compared to the rate and/or amount of cell plasma membrane repair in a cell not contacted with the candidate compound, provides an indication of the efficacy of a candidate compound's modulation of cell plasma membrane repair in the cell. Accordingly, one can monitor the rate and amount of cell plasma membrane repair (e.g. using an assay endpoint) to determine the efficacy of a candidate compound for modulation of cell plasma membrane repair. Thus, using the assays of the invention, one can identify compounds for use in the prevention and/or treatment of cell plasma membrane repair-associated disorders, diseases and conditions associated with abnormal cell plasma membrane repair, which include, but are not limited to: muscular dystrophy (e.g., Limb-girdle muscular dystrophy type 2B (LGMD2B), Miyoshi myopathy); otoferlin-associated deafness; a metabolic disorder (e.g. rhabdomyositis); calpain mutation-associated disease [e.g. Limb-girdle muscular dystrophy type 2A (LGMD2A)]; caveolin mutation-associated disease [e.g. Limb-girdle muscular dystrophy type 1C (LGMD1C)]; lysosomal storage disorders (e.g. Chediak-Higashi Syndrome); muscle cell trauma, or other cell trauma. In some embodiments of the invention, muscle cell trauma is exercise-associated muscle cell trauma or injury-associated muscle cell trauma.

It will be understood that a candidate compound that is identified as a modulating agent may be identified as increasing or enhancing cell plasma membrane repair, as evidenced by the results of assay endpoint determinations. Thus, in some embodiments, an increase in expression of a molecule that is specifically expressed in repaired plasma membrane (e.g. Lamp1) or by a decrease in the entry of an exogenous membrane impermeant molecule such as dextran or an impermeant toxin such as Gelonin or Granzyme B. Additionally, an increase in the release of enzymes such as LDH or β-hexosaminidase may indicate enhancement of cell plasma membrane repair by a compound. An increase in the assay endpoint results (and by extension, an increase in the rate and/or level of cell plasma membrane repair) may be any significant increase from the assay endpoint results in a control. The increase in the rate and/or amount of cell plasma membrane repair may mean an increase from zero cell plasma membrane repair or may be an increase from a control level of cell plasma membrane repair a higher level of cell plasma membrane repair. It will be understood that candidate agents and methods to decrease or increase the rate and/or amount of cell plasma membrane repair may be useful for research purposes, e.g. for making cell and/or animal models of disease conditions.

After incubation with a candidate modulatory compound the rate and/or amount of cell plasma membrane repair may be detected by any convenient method available to the user. One method of detection that is useful in the methods of the invention is the use of polypeptides (e.g. antibodies) that specifically bind to a marker of an assay endpoint. Detection may be effected in any convenient way for the assays of the invention. For example, an antibody may be coupled (directly or indirectly) to a detectable label. For cell-based assays, one of the assay components may comprise, or be coupled to, a detectable label.

The invention, in some aspects, includes the use of agents (e.g., antibodies or antigen-binding fragments thereof) to determine the presence and/or amount of marker molecules that are used to assess assay endpoints and determine the rate and/or amount of cell plasma membrane repair in the assays of the invention. As used herein, the term “antibodies” includes antibodies or antigen-binding fragments thereof. Antibodies of the invention can be identified and prepared that bind specifically to a marker molecule used in an assay endpoint of the invention.

As used herein, “binding specifically to” means capable of distinguishing the identified material from other materials sufficient for the purpose to which the invention relates. Thus, “binding specifically to” a marker useful in an assay endpoint of the invention means the ability to bind to and distinguish these molecules from other proteins. The antibodies and antigen-binding fragments thereof of the invention can be used for the assay of assay endpoints using known methods including, but not limited to, enzyme linked immunosorbent (ELISA) assays, immunoprecipitations, and Western blots.

The antibodies of the present invention may be prepared by any of a variety of methods, including administering protein, fragments of protein, cells expressing the protein or fragments thereof and the like to an animal to induce polyclonal antibodies. The production of monoclonal antibodies is according to techniques well known in the art. As detailed herein, such antibodies or antigen-binding fragments thereof may be used for example to identify the presence or level of a marker useful in an assay endpoint of the invention as an indication of the efficacy of a candidate compound for modulating the rate and/or amount of cell plasma membrane repair in a cell. The antibodies of the invention include monoclonal and polyclonal antibodies.

Antibodies also may be coupled to specific labeling agents, for example, for imaging of cells and tissues with according to standard coupling procedures. Labeling agents include, but are not limited to, fluorophores, chromophores, enzymatic labels, radioactive labels, etc. Other labeling agents useful in the invention will be apparent to one of ordinary skill in the art.

Thus, antibodies and/or antigen-binding fragments thereof are useful in methods of the invention. With respect to the antibodies and antigen-binding fragments thereof, as is well known in the art, only a small portion of an antibody molecule, the paratope, is involved in the binding of the antibody to its epitope (see, in general, Clark, W. R. (1986) The Experimental Foundations of Modem Immunology, Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd Fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, W. R. (1986) The Experimental Foundations of Modem Immunology, Wiley & Sons, Inc., New York; Roitt, I. (1991) Essential Immunology, 7th Ed., Blackwell Scientific Publications, Oxford). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205.

Thus, for example, PCT International Publication Number WO 92/04381 teaches the production and use of murine RSV antibodies in which at least a portion of the murine FR regions have been replaced by FR regions of human origin. Such antibodies, including fragments of intact antibodies with antigen-binding ability, are often referred to as “chimeric” antibodies. Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)2, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or nonhuman sequences. The present invention also includes so-called single chain antibodies.

Thus, the invention involves polypeptides of numerous size and type that bind specifically to a marker molecule useful in an assay endpoint of the invention. In some embodiments, polypeptides that bind specifically to a marker molecule useful in an assay endpoint of the invention are also useful to determine the efficacy of a candidate compound to modulate cell plasma membrane repair. The polypeptides may be derived also from sources other than antibody technology. For example, such polypeptide-binding agents can be provided by degenerate peptide libraries, which can be readily prepared in solution, in immobilized form or as phage display libraries. Combinatorial libraries also can be synthesized of peptides containing one or more amino acids. Libraries further can be synthesized of peptoids and non-peptide synthetic moieties.

In addition to the identification and assessment of cell plasma membrane repair-associated compounds, the methods of the invention are also useful to assess procedures and methods for treating cell plasma membrane-associated diseases, disorders, and conditions. In some embodiments the methods of the invention can be used to assess a cell modification, for example a genetic modification of a cell. An example, although not intended to be limiting, of a treatment whose effects and efficacy can be tested using the provided methods is genetic modification of cells or tissues to treat plasma membrane repair-associated disorders and diseases. In some embodiments, dystrophic muscle cells may be genetically modified by the addition of a gene that encodes a protein that is lacking from or is defective in the cells with the disorder. Following the genetic modification, the assays and assay endpoints of the invention can be used to compare the plasma membrane repair status of the modified cells versus unmodified cells (e.g. control cells) as a determination of the effect of the genetic modification on the cell plasma membrane repair (rate and/or amount).

Some modifications may increase the repair of the plasma membrane relative to the repair of the plasma membrane in a cell that does not have the genetic modification and therefore the modification will be an enhancer of plasma membrane repair. Alternatively, some modifications may decrease the repair of the plasma membrane relative to the repair of the plasma membrane in a cell that does not have the genetic modification and therefore will be an inhibitor of plasma membrane repair.

In some embodiments of the invention, the genetic modification is the delivery of a nucleic acid into the cell. The nucleic acid can be DNA, RNA, cDNA, etc. In preferable embodiments, the nucleic acid delivered into a cell is a cDNA. The cDNA can be cDNA of a gene that is involved in cell plasma membrane repair. For example, in some embodiments, the cDNA is a dysferlin cDNA.

In some embodiments of the invention, cDNA clones of the dysferlin gene that include different domains of the dysferlin gene can be produced and delivered to muscle cells using standard methods. The methods of the invention can then be used to assess the effect of the individual clones on plasma membrane repair in the cells. The amount and/or rate of repair of the plasma membrane in cell samples to which a clone has been delivered can be compared to the amount and/or rate of plasma membrane repair in a control cell sample (e.g. in a sample of cells absent a cDNA clone). The comparison will permit assessment of the effect of various cDNA clones on plasma membrane repair. The methods of the invention also can be used to distinguish functional versus non-functional clones, which provides information on dysferlin gene domains and their function and importance in plasma membrane repair.

Those of ordinary skill will recognize that the methods of the invention can be used to assess additional genes and to elucidate their functions in plasma membrane repair. Thus, the methods of the invention are useful to assess a candidate method of treating cell plasma membrane repair-associated disorders and also to determine the effectiveness of the therapeutic methods on cell plasma membrane repair-associated disorders and diseases. The aspects of the invention described herein relating to the assessment of candidate cell plasma membrane repair-modulating compounds are also applicable to assess methods of therapy for cell plasma membrane repair-associated diseases and/or disorders.

Kits

Some aspects of the invention include kits for assaying for compounds that modulate rate and/or amount of cell plasma membrane repair and kits to identify cells that have a plasma membrane repair-associated disorder. Kits of the invention may include one or more floating pins, which may be a component of a pin tool. Kits may also include solutions and materials for cleaning, maintaining, and using a floating pin and/or floating pin tool, including wash solutions, wash reservoirs, cleaning solutions, cleaning brushes, pin tool storage containers, replacement pins, replacement tubes, and pin blotting materials. A kit of the invention may also include control cells, solutions or molecules for use in the assays of the invention.

A kit of the invention may also include exogenous marker molecules, e.g. molecules that can be added to the environment of the cell as a measure of plasma membrane repair status. Examples of exogenous markers that may be included in a kit of the invention, although not intended to be limiting are: dextran, detectibly labeled dextran, cell plasma membrane integrity markers, trypan blue, cell plasma membrane impermeable toxins (e.g. Gelonin or Granzyme B etc.). The kits of the invention may also include solutions and materials for detecting markers. For example kits of the invention may include materials for determining the presence of Lamp1, lactate dehydrogenase (LDH) and/or β-hexosaminidase. In addition, detectible labels may be included in kits of the invention, as may instructions for labeling markers with detectable labels. Kits of the invention may also include components with which to test cell viability.

Kits of the invention may also include molecules that bind to markers as used in invention. The binding molecules may be antibodies or antigenic-fragments thereof and may be detectably labeled. The kit may also include materials for processing using procedures well known to those of skill in the art, to assess the presence and/or amount of a marker molecule as described herein. For example, procedures may include, but are not limited to, contact with a secondary antibody, or other method that indicates the presence of a marker.

Kits of the invention may include cells including control and/or test cells. Kits may also include vessels for cell growth including, but not limited to multi-well plates and may include solutions and materials for cell culture.

In some embodiments of the invention a kit may include a robotic apparatus for manipulating a pin tool robotically. The VP903a pin tool robot from V&P Scientific is an example of a type of pin tool robot that can be sued in the methods of the invention and may be provided in a kit of the invention.

The foregoing kits may also include instructions or other printed material on how to use the various components of the kits for identifying compounds that modulate cell plasma membrane repair or for identifying cells with a plasma membrane repair-associated disorder.

The methods of the invention can be used to screen or identify various compounds that are useful to modulate plasma membrane repair. The rate and/or level of plasma membrane repair may be increased, e.g., for prevention and/or treatment of muscular dystrophy (e.g., Limb-girdle muscular dystrophy type 2B (LGMD2B), Miyoshi myopathy); otoferlin-associated deafness; a metabolic disorder (e.g. rhabdomyositis); calpain mutation-associated disease [e.g. Limb-girdle muscular dystrophy type 2A (LGMD2A)]; caveolin mutation-associated disease [e.g. Limb-girdle muscular dystrophy type 1C (LGMD1C)]; lysosomal storage disorders (e.g. Chediak-Higashi Syndrome); and cell trauma (e.g. muscle or other cell trauma) using a compound identified through the methods of the invention as increasing plasma membrane repair. In some embodiments of the invention, exercise-associated muscle cell trauma and/or injury-associated muscle cell trauma, can be treated using compounds that modulate cell plasma membrane repair identified using the methods of the invention.

The invention includes in some aspects the use of compounds that modulate plasma membrane repair that are identified using the assays of the invention, in therapeutic methods for the prevention and treatment of conditions associated with abnormal plasma membrane repair. These methods of the invention include administration of plasma membrane repair-modulating compounds, to increase the rate and/or level of plasma membrane repair in cells or tissues. In general, the treatment methods of the invention involve administering an agent to modulate the rate and/or level of plasma membrane repair.

Methods of Treatment

In certain embodiments, the method for treating a subject with a disorder characterized by abnormal plasma membrane repair involves administering to the subject an effective amount of a candidate compound identified through a method or assay of the invention. Various techniques may be employed for introducing plasma membrane repair-modulating compounds of the invention to cells or tissues, depending on whether the compounds are introduced in vitro or in vivo in a host. In some embodiments, the plasma membrane repair-modulating compounds target muscle cells and/or tissues. Thus, the plasma membrane repair-modulating compounds can be specifically targeted to muscle tissue (e.g. muscle cells) using various delivery methods, including, but not limited to: administration to muscle tissue, the addition of targeting molecules to direct the compounds of the invention to muscle cells and/or tissues. Additional methods to specifically target molecules and compositions of the invention to muscle tissues are known to those of ordinary skill in the art.

In some embodiments of the invention, a plasma membrane repair-modulating compound of the invention may be delivered in the form of a delivery complex. The delivery complex may deliver the plasma membrane repair-modulating compound into any cell type, or may be associated with a molecule for targeting a specific cell type. Examples of delivery complexes include a plasma membrane repair-modulating compound of the invention associated with: a sterol (e.g., cholesterol), a lipid (e.g., a cationic lipid, virosome or liposome), or a target cell specific binding agent (e.g., an antibody, including but not limited to monoclonal antibodies, or a ligand recognized by target cell-specific receptor). Some delivery complexes may be sufficiently stable in vivo to prevent significant uncoupling prior to internalization by the target cell. However, the delivery complex can be cleavable under appropriate conditions within the cell so that the plasma membrane repair-modulating compound is released in a functional form.

An example of a targeting method, although not intended to be limiting, is the use of liposomes to deliver a plasma membrane repair-modulating compound of the invention into a cell. Liposomes may be targeted to a particular tissue, such as muscle or fibroblast cells, by coupling the liposome to a specific ligand such as a monoclonal antibody, sugar, glycolipid, or protein. Such proteins include proteins or fragments thereof specific for a particular cell type, antibodies for proteins that undergo internalization in cycling, proteins that target intracellular localization and enhance intracellular half life, and the like.

Liposomes are commercially available from Invitrogen Corporation (Carlsbad, Calif.), for example, as LIPOFECTIN® and LIPOFECTACE®, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications.

When administered, the plasma membrane repair-modulating compounds (also referred to herein as plasma membrane repair-modulating compositions and/or pharmaceutical compounds) of the present invention are administered in pharmaceutically acceptable preparations. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically acceptable salts thereof and are not excluded from the scope of the invention. Such pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts. Preferred components of the composition are described above in conjunction with the description of the pharmacological agents and/or compositions of the invention.

A plasma membrane repair modulating compound or composition may be combined, if desired, with a pharmaceutically acceptable carrier. The term “pharmaceutically acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the pharmacological agents of the invention, and with each other, in a manner such that there is no interaction which would substantially impair the desired pharmaceutical efficacy.

The plasma membrane repair-modulating compositions may contain suitable buffering agents, as described above, including: acetate, phosphate, citrate, glycine, borate, carbonate, bicarbonate, hydroxide (and other bases) and pharmaceutically acceptable salts of the foregoing compounds. The pharmaceutical compositions also may contain, optionally, suitable preservatives, such as: benzalkonium chloride; chlorobutanol; parabens and thimerosal.

The therapeutics of the invention can be administered by any conventional route including injection or by gradual infusion over time. Various modes of administration will be known to one of ordinary skill in the art which effectively deliver the pharmacological agents of the invention to a desired tissue, cell, or bodily fluid. The administration methods include: topical, intravenous, oral, inhalation, intracavity, intrathecal, intrasynovial, buccal, intraperitoneal, sublingual, intranasal, transdermal, intravitreal, subcutaneous, intramuscular and intradermal administration. The invention is not limited by the particular modes of administration disclosed herein. Standard references in the art (e.g., Remington: The Science and Practice of Pharmacy, A. R. Gennaro, Editor, 20^{th} edition, 2000) provide modes of administration and formulations for delivery of various pharmaceutical preparations and formulations in pharmaceutical carriers. Other protocols which are useful for the administration of pharmacological agents of the invention will be known to one of ordinary skill in the art, in which the dose amount, schedule of administration, sites of administration, mode of administration (e.g., intra-organ) and the like vary from those presented herein.

The therapeutic compositions may conveniently be presented in unit dosage form and may be prepared by any of the methods well known in the art of pharmacy. All methods include the step of bringing the compounds into association with a carrier that constitutes one or more accessory ingredients. In general, the compositions are prepared by uniformly and intimately bringing the therapeutic agent into association with a liquid carrier, a finely divided solid carrier, or both, and then, if necessary, shaping the product.

Compositions suitable for parenteral administration conveniently comprise a sterile aqueous preparation of the therapeutic agent, which is preferably isotonic with the blood of the recipient. This aqueous preparation may be formulated according to known methods using those suitable dispersing or wetting agents and suspending agents. The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono or diglycerides. In addition, fatty acids such as oleic acid find use in the preparation of injectables. Carrier formulations suitable for administration of a plasma membrane repair-modulating composition or compound can be found in Remington: The Science and Practice of Pharmacy, A. R. Gennaro, Editor, 20^{th} edition, 2000).

Compositions suitable for oral administration may be presented as discrete units such as capsules, cachets, tablets, or lozenges, each containing a predetermined amount of the therapeutic agent. Other compositions include suspensions in aqueous liquors or non-aqueous liquids such as a syrup, an elixir, or an emulsion.

The invention provides a composition of the above-described agents for use as a medicament, methods for preparing the medicament and methods for the sustained release of the medicament in vivo. Delivery systems can include time-release, delayed release or sustained release delivery systems. Such systems can avoid repeated administrations of the therapeutic agent of the invention, increasing convenience to the subject and the physician. Many types of release delivery systems are available and known to those of ordinary skill in the art. They include polymer-based systems such as polylactic and polyglycolic acid, poly(lactide-glycolide), copolyoxalates, polyanhydrides, polyesteramides, polyorthoesters, polyhydroxybutyric acid, and polycaprolactone. Microcapsules of the foregoing polymers containing drugs are described in, for example, U.S. Pat. No. 5,075,109. Nonpolymer systems that are lipids including sterols such as cholesterol, cholesterol esters and fatty acids or neutral fats such as mono-, di- and tri-glycerides; phospholipids; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

In one particular embodiment, the preferred vehicle is a biocompatible microparticle or implant that is suitable for implantation into the mammalian recipient. Exemplary bioerodible implants that are useful in accordance with this method are described in PCT International application no. PCT/US95/03307 (Publication No. WO 95/24929, entitled “Polymeric Gene Delivery System”). PCT/US95/03307 describes a biocompatible, preferably biodegradable polymeric matrix for containing an exogenous gene under the control of an appropriate promoter. The polymeric matrix is used to achieve sustained release of the exogenous gene in the patient. In accordance with the instant invention, the compound(s) of the invention is encapsulated or dispersed within a biocompatible, preferably biodegradable polymeric matrix, such as those disclosed in PCT/US95/03307. The polymeric matrix may be in the form of a microparticle such as a microsphere (wherein the compound is dispersed throughout a solid polymeric matrix) or a microcapsule (wherein the compound is stored in the core of a polymeric shell). Other forms of the polymeric matrix for containing the compounds of the invention include films, coatings, gels, implants, and stents. The size and composition of the polymeric matrix device is selected to result in favorable release kinetics in the tissue into which the matrix device is implanted. The size of the polymeric matrix device further is selected according to the method of delivery that is to be used. The polymeric matrix composition can be selected to have both favorable degradation rates and also to be formed of a material that is bioadhesive, to further increase the effectiveness of transfer when the device is administered to a vascular surface. The matrix composition also can be selected not to degrade, but rather, to release by diffusion over an extended period of time.

Both non-biodegradable and biodegradable polymeric matrices can be used to deliver agents of the invention of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

In general, the agents of the invention are delivered using the bioerodible implant by way of diffusion, or more preferably, by degradation of the polymeric matrix. Exemplary synthetic polymers that can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. Bioadhesive polymers of particular interest include bioerodible hydrogels described by H. S. Sawhney, C. P. Pathak and J. A. Hubell in Macromolecules, 1993:26:581-587, the teachings of which are incorporated herein by reference: polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Use of a long-term sustained release implant may be particularly suitable for treatment of established neurological disorder conditions as well as subjects at risk of developing a neurological disorder. “Long-term” release, as used herein, means that the implant is constructed and arranged to deliver therapeutic levels of the active ingredient for at least 7 days, and preferably at least 30-60 days or more. The implant may be positioned at or near the site of the cell damage, for example at or near a damaged muscle etc. The implant may also be placed at or near other tissues or regions affected by or involved in the plasma membrane repair-associated disease or disorder. Long-term sustained release implants are well known to those of ordinary skill in the art and include some of the release systems described above.

Some embodiments of the invention include methods for treating a subject to reduce the risk of a disorder associated with abnormal plasma membrane repair. The methods involve selecting and administering to a subject who is known to have, is suspected of having, or is at risk of having an abnormal plasma membrane repair, a plasma membrane repair-modulating compound for treating the disorder. Preferably, the plasma membrane repair-modulating compound is a compound for increasing plasma membrane repair and is administered in an amount effective to increase the rate and/or amount of plasma membrane repair.

Another aspect of the invention involves reducing the risk of a disorder associated with abnormal plasma membrane repair, by the use of treatments and/or medications to modulate plasma membrane repair thereby reducing, for example, the subject's risk of a plasma membrane repair-associated disorder.

In a subject determined to have a plasma membrane repair-associated disorder, an effective amount of a plasma membrane repair-modulating compound is that amount effective to modulate plasma membrane repair in a subject and therefore increase plasma membrane repair in the subject. For example, in the case of muscle injury (e.g. a sports injury) an effective amount may be an amount that increases the speed and/or amount of plasma membrane repair.

A response to a prophylatic and/or treatment method of the invention can, for example, also be measured by determining the physiological effects of the treatment or medication, such as the decrease or lack of disease symptoms following administration of the treatment or pharmacological agent. Other assays will be known to one of ordinary skill in the art and can be employed for measuring the level of the response. For example, the behavioral and neurological diagnostic methods that are used to ascertain the likelihood that a subject has a plasma membrane repair-associated disorder or disease, and to determine the putative stage of the disease can be used to ascertain the level of response to a prophylactic and/or treatment method of the invention. The amount of a treatment may be varied for example by increasing or decreasing the amount of a therapeutic composition, by changing the therapeutic composition administered, by changing the route of administration, by changing the dosage timing and so on. The effective amount will vary with the particular condition being treated, the age and physical condition of the subject being treated, the severity of the condition, the duration of the treatment, the nature of the concurrent therapy (if any), the specific route of administration, and other factors within the knowledge and expertise of the health practitioner.

The factors involved in determining an effective amount are well known to those of ordinary skill in the art and can be addressed with no more than routine experimentation. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

The therapeutically effective amount of a pharmacological agent of the invention is that amount effective to modulate plasma membrane repair and reduce, prevent, ameliorate, or eliminate a plasma membrane repair-associated disorder. Additional tests useful for monitoring the onset, progression, and/or remission, of a plasma membrane repair-associated disorder such as those described above herein, are well known to those of ordinary skill in the art. As would be understood by one of ordinary skill, for some disorders (e.g. muscular dystrophy, muscle injury) an effective amount would be the amount of a pharmacological agent identified using an assay of the invention that increases the levels and/or rate of plasma membrane repair to a level and/or rate that diminishes the disorder, as determined by the aforementioned tests. The methods of the invention can be used to select a method of treating a plasma membrane repair-associated disorder or disease and to determine effective amounts of therapeutic compounds or other treatments for cells, tissues, and/or subjects with a plasma membrane repair associated disease and/or disorder.

In the case of treating a particular disease or condition the desired response is inhibiting the progression of the disease or condition. This may involve only slowing the progression of the disease temporarily, although more preferably, it involves halting the progression of the disease permanently. This can be monitored by routine diagnostic methods known to one of ordinary skill in the art for any particular disease. The desired response to treatment of the disease or condition also can be delaying the onset or even preventing the onset of the disease or condition.

The pharmaceutical compositions used in the foregoing methods preferably are sterile and contain an effective amount of a pharmacological agent for producing the desired response in a unit of weight or volume suitable for administration to a patient.

The doses of pharmacological agents administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. The dosage of a pharmacological agent of the invention may be adjusted by the individual physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations daily, for one or more days.

Administration of pharmacological agents of the invention to mammals other than humans, e.g. for testing purposes or veterinary therapeutic purposes, is carried out under substantially the same conditions as described above. It will be understood by one of ordinary skill in the art that this invention is applicable to both human and animal diseases and disorders including plasma membrane repair-associated disorders of the invention. Thus, this invention is intended for use in husbandry and veterinary medicine as well as in human therapeutics.

The invention will be more fully understood by reference to the following examples. These examples, however, are merely intended to illustrate the embodiments of the invention and are not to be construed to limit the scope of the invention

### EXAMPLES

### Example 1

Methods

Membrane Repair Assay

Prior to the assay, muscle cells were plated at a density of 3000 cells/well. Cells were grown overnight in regular growth medium [Dulbecco's Modified Eagle Medium (DMEM), 15% Fetal Bovine Serum (FBS), Penicillin-Streptomycin Solution (Pen/Strep)] then induced to differentiate by switching to serum reduced medium (DMEM, 2.5% Horse Serum, Pen/Strep) for a further 48-72 hours. Wells then contained mostly myotubes.

The exact protocol for injury depended on the endpoint being used (see a-d below). Generally cells were either injured in the presence of a marker/active compound or a marker/compound was added at a specified time after injury.

The cell injury was made by from one to five applications of a customized pin tool (V&P Scientific, Inc. San Diego, Calif.). The number of contacts for the plasma membrane injury was dependent on extent of injury required. Depending on endpoint used, plates were either incubated overnight at 37° C., read immediately, or read after a specified time period.

a. Quantification of Immunofluorescent Markers on the Repaired Cell Surface.

Lamp1 is a lysosomal specific protein that we have shown to be selectively expressed in large quantities on the surface of repaired muscle cells. Cells prepared as described above were stained with Lamp1 in a 96-well plate as follows.

The media was removed from the 96-well plate and the cells were rinsed twice with Hank's Balanced Salt Solution (HBSS). The cells were injured as described above in the presence of HBSS and the dish placed on ice. 100 μl 4% (w/v) paraformaldehyde (PFA) was added to each well and incubated 10 minutes on ice. The cells were then rinsed three times with HBSS. The cells were blocked 20-30 minutes with 100 μl HBSS containing 10% (v/v) goat serum. The cells were incubated in the primary antibody one hour at 37° C. in a 1 in 100 dilution of Lamp1 monoclonal antibody 1D4Ba in HBSS/serum. 1D4Ba is a mouse-derived IgG2A type antibody (Developmental Studies Hybridoma Bank, University of Iowa, Iowa City, Iowa). The cells were then rinsed three times with HBSS. The cells were incubated in the secondary antibody 30 minutes at room temperature in a 1 in 40 dilution of Alexa-Fluor 488 nm (Molecular Probes, Eugene, Oreg.) goat anti-mouse secondary in HBSS/serum. The cells were rinsed twice with HBSS and the plate was read.

b. Quantification of Intracellular Enzymes Release into the Medium.

Membrane repair includes the fusion of intracellular vesicle systems with the cell surface membrane. As a result of this process the content of these vesicles is released to the extracellular environment of the cell. We were able to measure the release of these enzymes in both an absolute fashion and as a function of time after injury. Examples of enzymes that were measured are lactate dehydrogenase (LDH) and β-hexosaminidase. Cells prepared as described above were processed using a commercial LDH quantification kit (Roche, Basel, Switzerland) in a 96-well dish with the following procedure.

1. Quantification of LDH (Roche Kit)

Immediately before assay, the medium in each well was changed with 200 μl fusion medium (DMEM containing 2.5% (v/v) horse serum) added to each well. The cells were injured (as described above) and 100 μl aliquots of the cells were transferred to clear assay plate. 100 μl Reaction Mixture was prepared immediately before use and added to the cells. 250 μl of Catalyst (bottle 1; blue) and 11.25 ml of Dye Solution (bottle 2; red) were added to the cells. The dish was covered with foil and incubated in the dark for 30 min at room temperature. The cells were then read on an ELISA reader at 490 or 492 nm.

2. Quantification of β-Hexosaminidase Release Upon Cell Injury.

Preparation

Citrate/Phosphate Buffer (100 ml) was prepared by mixing: 44.1 ml of 0.2M Na₂HPO₄ (make 100 ml: 2.84 g in 100 ml ddH₂O) and 55.9 ml of 0.1M Citric Acid (make 100 ml: 1.92 g in 100 ml ddH₂O). The resulting buffer had a pH˜4.6.

Stop Solution (in 100 ml ddH₂O) was made by mixing 2 mM Na₂CO₃ (2.12 g) and 1.1 mM glycine (8.2577 g).

A 1 mM 4-Methylumbelliferyl solution in Citrate/Phosphate Buffer was made by adding 0.07588 g to 2 mls of citrate/phosphate buffer. This yielded 2 mls of solution, which is sufficient for four wells of a 96-well plate.

Assay

The medium in the 96-well plate was changed and the cells were allowed to sit 5 min. before 50 μl aliquots were removed from wells (Time=baseline) and the cells were either lysed or injured. If cells were injured, they were allowed to sit one minute and a 50 μl aliquot was taken (Time=0). The medium was changed and after 5 minutes in the fresh medium another 50 μl aliquot (Time=5) was taken. The medium was again changed and after 5 minutes in the fresh medium another 50 μl aliquot (Time=10) was taken. Each aliquot removed at each step was transferred a well in a labeled 96-well assay plate. 50 μl 4-methylumbelliferyl solution was added to each well of the assay plate and the plate was incubated 1 hr at 37° C. 100 μl Stop Solution was added to each well and the plate was covered with foil to protect from light. The plate was read immediately at 350 nm excitation, 448 nm emission.

c. Quantification of the Uptake of Fluorescent Molecules After Injury.

Dextrans are large molecular weight sugar molecules that can be labeled with fluorescent moieties and are commercially available from vendors such as Molecular Probes Inc. (Eugene, Oreg.). The dextran molecules are of such a size that they are unable to enter an intact cell. Membranes that have been compromised through injury allow entry of dextran molecules. The rate and amount of dextran influx into a cell was used as a measure of the efficiency of membrane repair.

Quantification of Dextran Uptake into Injured Cells

Cells were prepared as described above. The medium was aspirated from the wells and the cells were rinsed once with HBSS. 75 μl HBSS and 25 ml fluorescent dextran (e.g. Alexa 488 nm dextran) were added to the cells. The cells were injured and the dextran removed after 30 seconds. The cells were rinsed three times with HBSS and 100 μl 4% paraformaldehyde (PFA) was added and the cells incubated 10 min on ice. The cells were rinsed three times with HBSS and read at 488 nm.

d. Selective Toxicity of Cells with Injury Compromised Membranes.

Cytotoxic agents exist that only mediate their effects once inside a cell and at the same time lack the ability to cross the intact plasma membrane. Addition of one of these agents at a specific time after plasma membrane injury allowed the selective identification of cells with retarded membrane repair. Quantification was achieved using commercially available cell viability assays, for example, the LIVE/DEAD assay from Molecular Probes Inc., or WST Cytotoxicity assay from Pierce Inc. (Rockford, Ill.). Examples of toxins with these properties that have been used in the repair assays are Granzyme B and Gelonin.

Procedure for Selective Toxicity of Injured Cells

Cells were prepared in 96-well plates as described above. Fusion medium containing gelonin was added to each experimental well. The cells were injured as described above and the plates incubated overnight at 37° C. and 5% CO₂. A viability assay was performed (e.g. LIVE/DEAD viability/cytotoxicity assay from Molecular Probes inc.; or WST Cytotoxicity assay) according to Manufacturers' instructions.

Equivalents

Those skilled in the art will recognize, or be able to ascertain using no more than routine experimentation, many equivalents to the specific embodiments of the invention described herein. Such equivalents are intended to be encompassed by the following claims.

All references disclosed herein, including patent documents, are incorporated by reference in their entirety.

The terms and expressions that have been employed are used as terms of description and not of limitation, and there is no intention in the use of such terms and expressions of excluding any equivalents of the features shown and described or portions thereof, it being recognized that various modifications are possible within the scope of the invention.

Except where explicitly described otherwise, terms used in the singular also are meant to embrace the plural, and vice versa.

We claim:

1. A method for identifying compounds that modulate cell plasma membrane repair comprising:
injuring a plasma membrane of a cell by contacting the cell with a device that exerts force on the cell equal to about the weight of the device under gravity,
contacting the injured cell with a candidate compound, and
measuring the repair of the plasma membrane, wherein a difference in the repair of the plasma membrane relative to the repair of the plasma membrane in a control injured cell is an indication that the candidate compound modulates the repair of the plasma membrane,
optionally wherein a compound that increases the repair of the plasma membrane relative to the repair of the plasma membrane in a cell not contacted with the candidate compound is an enhancer of repair of the plasma membrane,
optionally wherein a compound that decreases the repair of the plasma membrane relative to the repair of the plasma membrane in a cell not contacted with the candidate compound is an inhibitor of repair of the plasma membrane,
optionally wherein the cell is in a multi-well plate,
optionally wherein the cell is initially contacted with the candidate compound before the time the cell is injured,
optionally wherein the cell is initially contacted with the candidate compound during or after the time the cell is injured,
optionally wherein thee device is a floating pin
optionally wherein the floating pin is one of a plurality of floating pins
optionally wherein the plurality of pins spatially correspond to the wells of a multi-well plate,
optionally wherein the plurality of floating pins is a floating pin tool,
optionally wherein the floating pin tool is robotically operated, optionally, wherein the floating pin tool is manually operated, and/or
optionally wherein repair is measured with a microplate reader.

2-3. (canceled)

4. The method of claim 1, wherein the cell is a cultured cell.

5. (canceled)

6. The method of claim 1, wherein the cell is a muscle cell.

7. The method of claim 1, wherein the cell is a fibroblast.

8-16. (canceled)

17. The method of claim 1, wherein the step of measuring the repair comprises contacting the cell with a toxin and determining cell death and/or cell viability,
optionally wherein the toxin is a cell-impermeable toxin, and/or
optionally wherein the cell-impermeable toxin is Gelonin or Granzyme B.

18-19. (canceled)

20. The method of claim 1, wherein the step of measuring the repair comprises determining the release of a molecule from the cell,
optionally wherein the molecule released from the cell is an enzyme, and/or
optionally wherein the enzyme is lactate dehydrogenase (LDH) or β-hexosaminidase.

21-22. (canceled)

23. The method of claim 1, wherein the step of measuring the repair comprises determining the entry into the cell of a molecule that does not enter the cell in the absence of a plasma membrane injury,
optionally wherein the molecule that does not enter the cell in the absence of a plasma membrane injury is dextran,
optionally wherein the molecule that does not enter the cell in the absence of a plasma membrane injury is detectably labeled,
optionally wherein the detectable label is a fluorescent label, and/or
optionally wherein the fluorescently labeled molecule is dextran.

24-27. (canceled)

28. The method of claim 1, wherein the step of measuring the repair comprises determining a marker that is selectively expressed by a cell with a repaired cell plasma membrane, and
optionally wherein the marker that is selectively expressed by the cell is Lamp1.

29. (canceled)

30. A method for identifying abnormal cell plasma membrane repair in a cell comprising:
injuring a plasma membrane of a sample cell by contacting the sample cell with a device that exerts force on the sample cell equal to about the weight of the device under gravity, and
measuring the repair of the sample cell plasma membrane, wherein a difference in the repair of the sample cell plasma membrane relative to the repair of a plasma membrane in a injured control cell is an indication of abnormal cell plasma membrane repair in the sample cell,
optionally wherein the muscle cell trauma is exercise-associated muscle trauma or injury associated muscle-cell trauma,
optionally wherein the sample cell is modified,
optionally wherein the modification of the sample cell is a genetic modification or a chemical modification,
optionally wherein the sample cell is from a subject having or suspected of having a muscle disorder or disease,
optionally wherein the sample cell is a cell that has been treated with a compound to alter plasma membrane repair,
optionally wherein the sample cell is from a subject that has been or is being treated for a plasma-membrane repair-associated disease or disorder,
optionally wherein the sample cell is a cultured cell,
optionally wherein the sample cell is a muscle cell, optionally, wherein the cell is in a multi-well plate,
optionally wherein the cell is initially contacted with the candidate compound before the time the cell is injured,
optionally wherein the cell is initially contacted with the candidate compound during or after the time the cell is injured,
optionally wherein the device is a floating pin
optionally wherein the floating pin is one of a plurality of floating pins,
optionally wherein the plurality of pins spatially correspond to the wells of a multi-well plate,
optionally wherein the plurality of floating pins is a floating pin tool,
optionally wherein the floating pin tool is robotically operated,
optionally wherein the floating pin tool is manually operated, and/or
optionally wherein repair is measured with a microplate reader.

31. The method of claim 30, wherein the sample cell is a cell suspected of having a plasma membrane repair-associated disorder,
optionally wherein the plasma membrane repair-associated disorder is a muscular dystrophy (e.g., Limb-girdle muscular dystrophy type 2B (LGMD2B), Miyoshi myopathy); otoferlin-associated deafness; a metabolic disorder (e.g. rhabdomyositis); calpain mutation-associated disease [e.g. Limb-girdle muscular dystrophy type 2A (LGMD2A)]; caveolin mutation-associated disease [e.g. Limb-girdle muscular dystrophy type 1C (LGMD1C)]; lysosomal storage disorders (e.g. Chediak-Higashi Syndrome); muscle cell trauma, or other cell trauma.

32-41. (canceled)

42. The method of claim 30, wherein the step of measuring the repair comprises contacting the cell with a toxin and determining cell death and/or cell viability,
optionally wherein the toxin is a cell-impermeable toxin, and/or
optionally wherein the cell-impermeable toxin is Gelonin or Granzyme B.

43-44. (canceled)

45. The method of claim 30, wherein the step of measuring the repair comprises determining the release of a molecule from the cell,
optionally wherein the molecule released from the cell is an enzyme, and/or
optionally wherein the enzyme is lactate dehydrogenase (LDH) or β-hexosaminidase.

46-47. (canceled)

48. The method of claim 30, wherein the step of measuring the repair comprises determining the entry into the cell of a molecule that does not enter the cell in the absence of a plasma membrane injury,
optionally wherein the molecule that does not enter the cell in the absence of a plasma membrane injury is dextran,
optionally wherein the molecule that does not enter the cell in the absence of a plasma membrane injury is detectably labeled,
optionally wherein the detectable label is a fluorescent label, and/or
optionally wherein the fluorescently labeled molecule is dextran.

49-52. (canceled)

53. The method of claim 30, wherein the step of measuring the repair comprises determining a marker that is selectively expressed by a cell with a repaired cell plasma membrane,
optionally wherein the marker that is selectively expressed by the cell is Lamp1.

54-63. (canceled)

64. A method of monitoring the onset, progression, or regression of a plasma-membrane repair-associated disorder in a subject comprising,
obtaining a first cell sample from a subject,
injuring the plasma membrane in the first cell sample by contacting the cell sample with a device that exerts force on the cell plasma membrane equal to about the weight of the device under gravity, and
measuring the repair of the plasma membrane in the first cell sample,
obtaining a second cell sample from the subject at a time subsequent to the time the first cell sample was obtained,
injuring a plasma membrane of the second cell sample by contacting the cell sample with the device that exerts force on the cell plasma membrane equal to about the weight of the device under gravity, and
measuring the repair of the second cell sample plasma membrane,
wherein a difference in the repair of the first cell sample plasma membrane relative to the repair of the second cells sample plasma membrane is an indication of the onset, progression, or regression of the plasma membrane repair-associated disorder in the subject,
optionally wherein the cell sample has been treated with a compound to alter plasma membrane repair,
optionally wherein the cell sample is from a subject that has been or is being treated for a plasma-membrane repair-associated disease or disorder,
optionally wherein the cell sample is cultured prior to being injured,
optionally wherein the cell sample comprises muscle cells,
optionally wherein the cell sample is in a multi-well plate,
optionally wherein the cell is initially contacted with the candidate compound before the time the cell is injured,
optionally wherein the cell is initially contacted with the candidate compound during or after the time the cell is injured,
optionally wherein the device is a floating pin,
optionally wherein the floating pin is one of a plurality of floating pins,
optionally wherein the plurality of pins spatially correspond to the wells of a multi-well plate,
optionally wherein the plurality of floating pins is a floating pin tool,
optionally wherein the floating pin tool is robotically operated,
optionally wherein the floating pin tool is manually operated, and/or
optionally wherein repair is measured with a microplate reader.

65. The method of claim 64, wherein the subject is undergoing treatment for a plasma membrane repair-associated disorder.

66. The method of claim 64, wherein the sample cell is from a subject having or suspected of having a muscle disorder or disease,
optionally wherein the plasma membrane repair-associated disorder or disease is a muscular dystrophy (e.g., Limb-girdle muscular dystrophy type 2B (LGMD2B), Miyoshi myopathy), otoferlin-associated deafness: a metabolic disorder (e.g. rhabdomyositis); calpain mutation-associated disease [e.g. Limb-girdle muscular dystrophy type 2A (LGMD2A)]; caveolin mutation-associated disease [e.g. Limb-girdle muscular dystrophy type 1C (LGMD1C)]; lysosomal storage disorders (e.g. Chediak-Higashi Syndrome); muscle cell trauma, or other cell trauma, and/or
optionally wherein the muscle cell trauma is exercise-associated muscle trauma or injury associated muscle-cell trauma.

67-73. (canceled)

74. The method of claim 64, wherein the step of measuring the repair comprises contacting the cell with a toxin and determining cell death and/or cell viability,
optionally wherein the toxin is a cell-impermeable toxin, and/or
optionally wherein the cell-impermeable toxin is Gelonin or Granzyme B.

75-76. (canceled)

77. The method of claim 64, wherein the step of measuring the repair comprises determining the release of a molecule from the cell,
optionally wherein the molecule released from the cell is an enzyme, and/or
optionally wherein the enzyme is lactate dehydrogenase (LDH) or β-hexosaminidase.

78-79. (canceled)

80. The method of claim 64, wherein the step of measuring the repair comprises determining the entry into the cell of a molecule that does not enter the cell in the absence of a plasma membrane injury,
optionally wherein the molecule that does not enter the cell in the absence of a plasma membrane injury is dextran,
optionally wherein the molecule that does not enter the cell in the absence of a plasma membrane injury is detectably labeled,
optionally wherein the detectable label is a fluorescent label, and/or
optionally wherein the fluorescently labeled molecule is dextran.

81-84. (canceled)

85. The method of claim 64, wherein the step of measuring the repair comprises determining a marker that is selectively expressed by a cell with a repaired cell plasma membrane,
optionally wherein the marker that is selectively expressed by the cell is Lamp1.

86. (canceled)

87-142. (canceled)
